(19) United States
(12) Patent Application Publication
(10) Pub. No.: US 2023/0357392 A1
(43) Pub. Date: Nov. 9, 2023

(54) Treatment Paradigm for an Anti-CD19 Antibody Therapy

(71) Applicant: MORPHOSYS AG, Planegg (DE)

(72) Inventors: Stefan HÄRTLE, Mammendorf (DE); Frank STRIEBEL, Munich (DE)

(21) Appl. No.: 18/087,068

(22) Filed: Dec. 22, 2022

(30) Foreign Application Priority Data
Dec. 22, 2021 (EP) 21216714.2

(51) Int. Cl. C07K 16/28; A61P 35/00; A61K 31/454

(52) U.S. Cl. CPC C07K 16/2803; A61P 35/00; A61K 31/454; A61K 2039/545

(57) ABSTRACT

The present disclosure provides anti-CD19 antibodies for use in the treatment of various cancers. The anti-CD19 antibody is administered to cancer patients in a specific dose or dosing regimen.

This patent application claims the benefit of priority from EP 2216714.2 filed Dec. 22, 2021, the content of which is incorporated by reference in its entirety.

### REFERENCE TO AN ELECTRONIC SEQUENCE LISTING

The contents of the electronic sequence listing (MOR0089US.xml); Size 19,195 bytes; and Date of Creation: Dec. 19, 2022) is herein incorporated by reference in its entirety.

### FIELD OF THE INVENTION

The present disclosure provides a treatment comprising an anti-CD19 antibody for use in the treatment of various cancers.

### BACKGROUND

B cells are lymphocytes that play a large role in the humoral immune response. They are produced in the bone marrow of most mammals, and represent 5-15% of the circulating lymphoid pool. The principal function of B cells is to make antibodies against various antigens, and are an essential component of the adaptive immune system. Because of their critical role in regulating the immune system, dysregulation of B cells is associated with a variety of disorders, such as cancer. These include lymphomas and leukemia such as non-Hodgkin's lymphoma (NHL), chronic lymphocytic leukemia (CLL), small lymphocytic lymphoma (SLL) and acute lymphoblastic leukemia (ALL).

NHL is a heterogeneous malignancy originating from lymphocytes. While the disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumors, which organizes NHL into precursor and mature B-cell or T-cell neoplasms. The Physician's Data Query is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell cancers (DLBL, DLBCL, or DLCL; where 40% of all newly diagnosed patients have diffuse large cell), Burkitt lymphoma, and mantle cell lymphoma.

In addition to NHL there are several types of leukemia that result from dysregulation of B cells.

Chronic lymphocytic leukemia (also known as “chronic lymphoid leukemia” or “CLL”), is a type of adult leukemia caused by an abnormal accumulation of B lymphocytes. In CLL, the malignant lymphocytes may look normal and mature, but they are not able to cope effectively with infection. CLL is the most common form of leukemia in adults. Men are twice as likely to develop CLL as women. However, the key risk factor is age. CLL is an incurable disease but progresses slowly in most cases. Many people with CLL lead normal and active lives for many years. Because of its slow onset, early-stage CLL is generally not treated since it is believed that early CLL intervention does not improve survival time or quality of life. Instead, the condition is monitored over time. Initial CLL treatments vary depending on the exact diagnosis and the progression of the disease. There are dozens of agents used for CLL therapy. Combination chemotherapy regimens such as FCR (fludarabine, cyclophosphamide and rituximab), and BR (Ibrutinib and rituximab) are effective in both newly-diagnosed and relapsed CLL. Allogeneic bone marrow (stem cell) transplantation is rarely used as a first-line treatment for CLL due to its risk.

Another type of leukemia is small lymphocytic lymphoma (SLL) that is considered a CLL variant that lacks the clonal lymphocytosis required for the CLL diagnosis, but otherwise shares pathological and immunophenotypic features (Campo et al., 2011). The definition of SLL requires the presence of lymphadenopathy and/or splenomegaly.

Moreover, the number of B lymphocytes in the peripheral blood should not exceed 5×10⁹/L. In SLL, the diagnosis should be confirmed by histopathologic evaluation of a lymph node biopsy whenever possible (Hallek et al., 2008).

Another type of leukemia, acute lymphoblastic leukemia, is characterized by the overproduction and continuous multiplication of malignant and immature white blood cells (also known as lymphoblasts) in the bone marrow, Acute lymphoblastic leukemia is most common in childhood with a peak incidence of 4-5 years of age.

The human CD19 molecule is a structurally distinct cell surface receptor expressed on the surface of human B cells, including, but not limited to, pre-B cells, B cells in early development (i.e., immature B cells), mature B cells through terminal differentiation into plasma cells, and malignant B cells. CD 19 is expressed by most pre-B acute lymphoblastic leukemias (ALL), non-Hodgkin's lymphomas, B cell chronic lymphocytic leukemias (CLL), small lymphocytic lymphomas (SLL), pro-lymphocytic leukemias, hairy cell leukemias, common acute lymphocytic leukemias, and some Null-acute lymphoblastic leukemias (Nadler et al, J. Immunol., 131:244-250 (1983), Loken et al, Blood, 70:1316-1324 (1987), Uckun et al, Blood, 71:13-29 (1988), Anderson et al, 1984. Blood, 63:1424-1433 (1984), Scheuermann, Leuk. Lymphoma, 18:385-397(1995)). The expression of CD19 on plasma cells further suggests it may be expressed on differentiated B cell tumors such as multiple myeloma, plasmacytomas, Waldenstrom's tumors (Grossbard et al., Br. J. Haematol, 102:509-15(1998); Treon et al, Semin. Oncol, 30:248-52(2003)). Therefore, the CD19 antigen is a target for immunotherapy in the treatment of various cancers, such as non-Hodgkin's lymphoma (NHL), chronic lymphocytic leukemia (CLL), small lymphocytic lymphoma (SLL) and/or acute lymphoblastic leukemia, including each of the subtypes described herein.

Tafasitamab (former names: MOR208 and XmAb®5574) is a humanized monoclonal antibody that targets the antigen CD19. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. Based on the L-MIND trial tafasitamab received accelerated approval from the US Food and Drug Administration (FDA) in July 2020 for use in combination with lenalidomide to treat adults with R/R DLBCL. The recommended dose of tafasitamab is 12 mg/kg, administered as intravenous (i.v.) infusion.

Despite recent discoveries and developments of several anti-cancer agents, due to poor prognosis for many types of cancers including CD19-expressing tumors, there is still a need for an improved method or therapeutic approach for treating such types of cancers.

### SUMMARY

The need was identified to optimize the tafasitamab dosing regimen to reduce the frequency of hospital/clinic visits for tafasitamab treated patients. Reducing the overall frequency of clinic visits by half is expected to reduce the patient burden and support long term treatment compliance. Furthermore, given the severity of disease, reduced hospital visits may result in less exposure to hospital infections in the already susceptible population.

The present disclosure provides a treatment paradigm for a therapy comprising an anti-CD19 antibody, wherein said anti-CD19 antibody is administered at a dose of at least 24 mg/kg.

In one aspect, the present disclosure relates to an anti-CD19 antibody for use in the treatment of a cancer wherein said anti-CD19 antibody is administered at a dose of at least 24 mg/kg.

In one aspect, the present disclosure relates to methods of treating a cancer comprising administering to a human subject in need of said treatment an anti-CD19 antibody, wherein said anti-CD19 antibody is administered at a dose of at least 24 mg/kg.

In one aspect, the present disclosure relates to an anti-CD19 antibody for use in the treatment of a cancer wherein said anti-CD19 antibody is administered at a dose of at least 24 mg/kg and wherein such dosing reduces the dosing frequency from once weekly to at least once every two weeks. In an embodiment the dosing frequency is reduced to once every two weeks. In an embodiment the dosing frequency is reduced to once every 4 weeks. In an embodiment the dosing frequency is reduced to once every 5 weeks. In an embodiment the dosing frequency is reduced to once every 6 weeks. In an embodiment the dosing frequency is reduced to once every 7 weeks. In an embodiment the dosing frequency is reduced to once every 8 weeks.

In one aspect, the present disclosure relates to an anti-CD19 antibody for use in the treatment of a cancer wherein said anti-CD19 antibody is administered at a dose of at least 24 mg/kg and wherein such dosing reduces the dosing frequency from once weekly to once every 4 weeks.

In one aspect, the present disclosure relates to an anti-CD19 antibody for use in the treatment of a cancer wherein said anti-CD19 antibody is administered at a dose of at least 24 mg/kg and wherein such dosing reduces the dosing frequency from once weekly to once every two weeks and to once every 4 weeks.

In one aspect, the present disclosure relates to an anti-CD19 antibody for use in the treatment of a cancer wherein said anti-CD19 antibody is administered at a dose of at least 24 mg/kg and wherein such dosing reduces the dosing frequency from once weekly (QW) to once every two weeks (Q2W) from Cycle 1 Day 15 (C1D15) onwards and from Q2W to once every 4 weeks (Q4W) from Cycle 4 Day 1 onwards.

In one aspect, the present disclosure relates to an anti-CD19 antibody for use in the treatment of a cancer wherein said anti-CD19 antibody is administered at a dose of at least 24 mg/kg and wherein such dosing reduces the dosing regimen by 20%, 30%, 40%, 50%, 60%, 70% or more of infusions in comparison to the administration of such anti-CD19 antibody at a dose of 12 mg/kg.

In one aspect, the present disclosure relates to methods of reducing the dosing frequency of an anti-CD19 antibody in the treatment of a cancer wherein said anti-CD19 antibody is administered at a dose of at least 24 mg/kg.

In one aspect, the present disclosure relates to methods of reducing the dosing frequency of an anti-CD19 antibody in the treatment of a cancer wherein said anti-CD19 antibody is administered at a dose of at least 24 mg/kg and wherein such dosing reduces the dosing frequency from once weekly to at least once every two weeks. In an embodiment the dosing frequency is reduced to once every 2 weeks. In an embodiment the dosing frequency is reduced to once every 4 weeks. In an embodiment the dosing frequency is reduced to once every 5 weeks. In an embodiment the dosing frequency is reduced to once every 6 weeks. In an embodiment the dosing frequency is reduced to once every 7 weeks. In an embodiment the dosing frequency is reduced to once every 8 weeks.

In one aspect, the present disclosure relates to methods of reducing the dosing frequency of an anti-CD19 antibody in the treatment of a cancer wherein said anti-CD19 antibody is administered at a dose of at least 24 mg/kg and wherein such dosing reduces the dosing frequency from once weekly to once every 4 weeks.

In one aspect, the present disclosure relates to methods of reducing the dosing frequency of an anti-CD19 antibody in the treatment of a cancer wherein said anti-CD19 antibody is administered at a dose of at least 24 mg/kg and wherein such dosing reduces the dosing frequency from once weekly to once every two weeks and to once every 4 weeks.

In one aspect, the present disclosure relates to methods of reducing the dosing frequency of an anti-CD19 antibody in the treatment of a cancer wherein said anti-CD19 antibody is administered at a dose of at least 24 mg/kg and wherein such dosing reduces the dosing frequency from once weekly (QW) to once every two weeks (Q2W) from Cycle 1 Day 15 (C1D15) onwards and from Q2W to once every 4 weeks (Q4W) from Cycle 4 Day 1 onwards.

In one aspect, the present disclosure relates to methods of reducing the dosing frequency of an anti-CD19 antibody in the treatment of a cancer wherein said anti-CD19 antibody is administered at a dose of at least 24 mg/kg and wherein such dosing reduces the dosing regimen by 20%, 30%, 40%, 50%, 60%, 70% or more of infusions in comparison to a the administration of such anti-CD19 antibody at a dose of 12 mg/kg.

In one aspect, the present disclosure relates to methods of reducing adverse effects of an anti-CD19 antibody for use in the treatment of a cancer at a dose of at least 24 mg/kg, wherein said anti-CD19 antibody is administered at a dose of 12 mg/kg prior to an increase to the dose of at least 24 mg/kg. In some embodiments, the anti-CD19 antibody is administered at a dose of 12 mg/kg for the first one, two or three administrations and wherein after such first one, two or three administrations the anti-CD19 antibody is administered at a dose of at least 24 mg/kg. In some embodiments, the anti-CD19 antibody is administered at a dose of at least 24 mg/kg once a week, once every two weeks or once every four weeks.

In one aspect, the present disclosure relates to methods of reducing adverse effects of an anti-CD19 antibody for use in the treatment of a cancer at a dose of at least 24 mg/kg, wherein said anti-CD19 antibody is administered at a dose of 12 mg/kg on day 1, day 4 and day 8 from the start of treatment and from day 15 the anti-CD19 antibody is administered at a dose of at least 24 mg/kg. In some embodiments, the anti-CD19 antibody is administered at a dose of at least 24 mg/kg once a week, once every two weeks or once every four weeks.

In some embodiments, the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In some embodiments, the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In some embodiments, the anti-CD19 antibody comprises a heavy chain variable region of

(SEQ ID NO: 7)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSS

and a light chain variable region of

(SEQ ID NO: 8)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIK.

In some embodiments, the anti-CD19 antibody has effector function. In another aspect the antibody or antibody fragment specific for CD19 has an enhanced effector function. In one embodiment the effector function is ADCC. In one embodiment the antibody or antibody fragment specific for CD19 has an enhanced ADCC activity. In a further embodiment the antibody or antibody fragment specific for CD19 comprises an Fc domain comprising an amino acid substitution at position S239 and/or 1332, wherein the numbering is according to the EU index as in Kabat. In a further embodiment the antibody or antibody fragment specific for CD19 comprises an Fc domain comprising an S239D amino acid substitution and an I332E amino acid substitution, wherein the numbering is according to the EU index as in Kabat.

In some embodiments, the anti-CD19 antibody comprises a heavy chain constant region of

(SEQ ID NO: 9)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPDVFLFPPKPKDTLMISRTPEVTCVVV
DVSHEDPEVQFNWYVDGVEVHNAKTKPREEQFNSTFRVVSVLTVVHQDW
LNGKEYKCKVSNKALPAPEEKTISKTKGQPREPQVYTLPPSREEMTKNQ
VSLTCLVKGFYPSDIAVEWESNGQPENNYKTTPPMLDSDGSFFLYSKLT
VDKSRWQQGNVFSCSVMHEALHNHYTQKSLSLSPGK.

In some embodiments, the anti-CD19 antibody comprises a light chain constant region of

(SEQ ID NO: 10)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some embodiments, the anti-CD19 antibody comprises a heavy chain constant region of

(SEQ ID NO: 9)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPDVFLFPPKPKDTLMISRTPEVTCVVV
DVSHEDPEVQFNWYVDGVEVHNAKTKPREEQFNSTFRVVSVLTVVHQDW
LNGKEYKCKVSNKALPAPEEKTISKTKGQPREPQVYTLPPSREEMTKNQ
VSLTCLVKGFYPSDIAVEWESNGQPENNYKTTPPMLDSDGSFFLYSKLT
VDKSRWQQGNVFSCSVMHEALHNHYTQKSLSLSPGK

and a light chain constant region of

(SEQ ID NO: 10)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some embodiments, the anti-CD19 antibody comprises a heavy chain region of

(SEQ ID NO: 11)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPDVF
LFPPKPKDTLMISRTPEVTCVVVDVSHEDPEVQFNWYVDGVEVHNAKTK
PREEQFNSTFRVVSVLTVVHQDWLNGKEYKCKVSNKALPAPEEKTISKT
KGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYT
QKSLSLSPGK

and a light chain region of

(SEQ ID NO: 12)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

In some embodiments, the anti-CD19 antibody is tafasitamab.

In some embodiments, the anti-CD19 antibody is administered intravenously.

In some embodiments, the anti-CD19 antibody is administered by intravenous infusion.

In some embodiments, the anti-CD19 antibody is administered by subcutaneous injection.

In some embodiments, the anti-CD19 antibody is administered subcutaneously.

In some embodiments, the cancer is a CD19 positive cancer.

In some embodiments, the cancer is a hematological malignancy.

In some embodiments, the cancer is a lymphoma or a leukemia.

In some embodiments, the cancer is a chronic lymphocytic leukemia or a non-Hodgkin's lymphoma.

In some embodiments, the cancer is a CD19 positive chronic lymphocytic leukemia or a CD19 positive non-Hodgkin's lymphoma.

In some embodiments, the cancer is a non-Hodgkin lymphoma. In some embodiments the human subject suffers from relapsed or refractory non-Hodgkin lymphoma. In some embodiments, human subject suffers from relapsed or refractory CD19-positive aggressive non-Hodgkin lymphoma. In some embodiments, the human subject suffers from relapsed or refractory CD19-positive aggressive non-Hodgkin lymphoma and has progressed on at least one prior treatment regimen. In some embodiments the cancer is follicular lymphoma (FL), marginal zone lymphoma (MZL), mantle cell lymphoma (MCL), Diffuse large B-cell lymphoma (DLBCL), or Burkitt lymphoma. In some embodiments the human subject suffers from relapsed or refractory Diffuse large B-cell lymphoma (r/r DLBCL).

In some embodiments, the anti-CD19 antibody is administered at a dose of at least 24 mg/kg wherein the anti-CD19 antibody is administered as an intravenous infusion. In some embodiments, said intravenous infusion is administered within at least 1.5 hours, at least 2 hours, at least 2.5 hours, at least 3 hours, at least 3.5 hours, at least 4 hours or at least 4.5 hours.

In some other embodiments, said intravenous infusion is administered within at least 1.5 to 2.5 hours, at least 2.5 to 3 hours, at least 2.5 to 3.5 hours, at least 3 to 4 hours, or at least 3.5 to 4.5 hours. In some other embodiments, said intravenous infusion is administered within 1.5 to 2.5 hours, 2 hours, 2.5 to 3 hours, 3 hours, 2.5 to 3.5 hours, 3 to 4 hours, or 3.5 to 4.5 hours. In some other embodiments, said intravenous infusion is administered with an infusion rate of at least 30 mL/h, at least 40 mL/h, at least 50 mL/h, at least 60 mL/h or at least 70 mL/h. In some other embodiments, said intravenous infusion is administered initially with an infusion rate of 30 mL/h, 40 mL/h, 50 mL/h, 60 mL/h or 70 mL/h for the first 30 minutes and then increased.

In some embodiments, the anti-CD19 antibody is administered at a dose of at least 24 mg/kg wherein the anti-CD19 antibody is administered as an intravenous infusion and wherein said intravenous infusion is administered within 1.5 to 2.5 hours. In some other embodiments, the anti-CD19 antibody is administered at a dose of at least 24 mg/kg wherein the anti-CD19 antibody is administered as an intravenous infusion and wherein said intravenous infusion is administered within 1.5 to 2 hours. In some other embodiments, the anti-CD19 antibody is administered at a dose of at least 24 mg/kg wherein the anti-CD19 antibody is administered as an intravenous infusion and wherein said intravenous infusion is administered within 2 hours.

In some embodiments, the anti-CD19 antibody is administered at a dose of at least 24 mg/kg wherein the anti-CD19 antibody is administered as an intravenous infusion and wherein said intravenous infusion is administered within 3 to 4.5 hours. In some other embodiments, the anti-CD19 antibody is administered at a dose of at least 24 mg/kg wherein the anti-CD19 antibody is administered as an intravenous infusion and wherein said intravenous infusion is administered within 3 to 4 hours. In some other embodiments, the anti-CD19 antibody is administered at a dose of at least 24 mg/kg wherein the anti-CD19 antibody is administered as an intravenous infusion and wherein said intravenous infusion is administered within 3.5 to 4 hours. In some other embodiments, the anti-CD19 antibody is administered at a dose of at least 24 mg/kg wherein the anti-CD19 antibody is administered as an intravenous infusion and wherein said intravenous infusion is administered within 3.5 to 4.5 hours. In some other embodiments, the anti-CD19 antibody is administered at a dose of at least 24 mg/kg wherein the anti-CD19 antibody is administered as an intravenous infusion and wherein said intravenous infusion is administered within 4 hours.

In some embodiments, the present disclosure relates to a method of treating a cancer in a human subject in need thereof, the method comprising administering to the human subject an anti-CD19 antibody, wherein said anti-CD19 antibody is administered at a dose of 12 mg/kg prior to an increase to the dose of at least 24 mg/kg (for example 24 mg/kg or 30 mg/kg). In some embodiments, the anti-CD19 antibody is administered at a dose of 12 mg/kg for the first one, two or three administrations and wherein after such first one, two or three administrations the anti-CD19 antibody is administered at a dose of at least 24 mg/kg (for example 24 mg/kg or 30 mg/kg). In some embodiments, the anti-CD19 antibody is administered at a dose of at least 24 mg/kg once a week, once every two weeks or once every four weeks.

In some embodiments, the present disclosure relates to a method of treating a cancer in a human subject in need thereof, the method comprising administering to the human subject an anti-CD19 antibody, wherein said anti-CD19 antibody is administered at a dose of 12 mg/kg on day 1, day 4 and day 8 from the start of treatment and from day 15 the anti-CD19 antibody is administered at a dose of at least 24 mg/kg. In some embodiments, the anti-CD19 antibody is administered at a dose of at least 24 mg/kg once a week, once every two weeks or once every four weeks.

In some embodiments, the present disclosure relates to a method of treating a cancer in a human subject in need thereof, the method comprising administering to the human subject an anti-CD19 antibody, wherein said anti-CD19 antibody is administered as an intravenous infusion at a dose of 12 mg/kg within 1.5 to 2.5 hours prior to an increase to the dose of at least 24 mg/kg (for example 24 mg/kg or 30 mg/kg) and wherein said increased dose of at least 24 mg/kg (for example 24 mg/kg or 30 mg/kg) is administered as an intravenous infusion within 3 to 4.5 hours. In some other embodiments, said increased dose of at least 24 mg/kg (for example 24 mg/kg or 30 mg/kg) is administered as an intravenous infusion within 3 to 4 hours. In some other embodiments, said increased dose of at least 24 mg/kg (for example 24 mg/kg or 30 mg/kg) is administered as an intravenous infusion within 3.5 to 4 hours. In some other embodiments, said increased dose of at least 24 mg/kg (for example 24 mg/kg or 30 mg/kg) is administered as an intravenous infusion within 3.5 to 4.5 hours. In some other embodiments, said increased dose of at least 24 mg/kg (for example 24 mg/kg or 30 mg/kg) is administered as an intravenous infusion within 4 hours.

In some embodiments, the anti-CD19 antibody is administered as an intravenous infusion at a dose of 12 mg/kg within 1.5 to 2.5 hours for the first one, two or three administrations and wherein after such first one, two or three administrations the anti-CD19 antibody is administered at a dose of at least 24 mg/kg (for example 24 mg/kg or 30 mg/kg) and wherein said increased dose of at least 24 mg/kg (for example 24 mg/kg or 30 mg/kg) is administered as an intravenous infusion within 3 to 4.5 hours. In some other embodiments, said increased dose of at least 24 mg/kg (for example 24 mg/kg or 30 mg/kg) is administered as an intravenous infusion within 3 to 4 hours. In some other embodiments, said increased dose of at least 24 mg/kg (for example 24 mg/kg or 30 mg/kg) is administered as an intravenous infusion within 3.5 to 4 hours. In some other embodiments, said increased dose of at least 24 mg/kg (for example 24 mg/kg or 30 mg/kg) is administered as an intravenous infusion within 3.5 to 4.5 hours. In some other embodiments, said increased dose of at least 24 mg/kg (for example 24 mg/kg or 30 mg/kg) is administered as an intravenous infusion within 4 hours. In some embodiments, the anti-CD19 antibody is administered at a dose of at least 24 mg/kg (for example 24 mg/kg or 30 mg/kg) once a week, once every two weeks or once every four weeks.

In some embodiments, the present disclosure relates to a method of treating a cancer in a human subject in need thereof, the method comprising administering to the human subject an anti-CD19 antibody, wherein said anti-CD19 antibody is administered as an intravenous infusion at a dose of 12 mg/kg within 1.5 to 2.5 hours prior to an increase to the dose of at least 24 mg/kg (for example 24 mg/kg or 30 mg/kg) and wherein said increased dose of at least 24 mg/kg (for example 24 mg/kg or 30 mg/kg) is administered as an intravenous infusion within 1.5 to 2.5 hours. In some other embodiments, said increased dose of at least 24 mg/kg (for example 24 mg/kg or 30 mg/kg) is administered as an intravenous infusion within 1.5 to 2 hours. In some other embodiments, said increased dose of at least 24 mg/kg (for example 24 mg/kg or 30 mg/kg) is administered as an intravenous infusion within 2 hours. In some other embodiments, said increased dose of at least 24 mg/kg (for example 24 mg/kg or 30 mg/kg) is administered as an intravenous infusion within 2 hours.

In some embodiments, the anti-CD19 antibody is administered as an intravenous infusion at a dose of 12 mg/kg within 1.5 to 2.5 hours for the first one, two or three administrations and wherein after such first one, two or three administrations the anti-CD19 antibody is administered at a dose of at least 24 mg/kg (for example 24 mg/kg or 30 mg/kg) and wherein said increased dose of at least 24 mg/kg (for example 24 mg/kg or 30 mg/kg) is administered as an intravenous infusion within 1.5 to 2.5 hours. In some other embodiments, said increased dose of at least 24 mg/kg (for example 24 mg/kg or 30 mg/kg) is administered as an intravenous infusion within 1.5 to 2 hours. In some other embodiments, said increased dose of at least 24 mg/kg (for example 24 mg/kg or 30 mg/kg) is administered as an intravenous infusion within 2 hours.

In some embodiments, the anti-CD19 antibody is administered as an intravenous infusion at a dose of 12 mg/kg within 1.5 to 2.5 hours for the first one, two and three administrations and wherein after such first one, two and three administrations the anti-CD19 antibody is administered at a dose of at least 24 mg/kg (for example 24 mg/kg or 30 mg/kg) and wherein for the first one, two and three administrations said increased dose of at least 24 mg/kg (for example 24 mg/kg or 30 mg/kg) is administered as an intravenous infusion within 3 to 4.5 hours and wherein for all subsequent administrations said increased dose of at least 24 mg/kg (for example 24 mg/kg or 30 mg/kg) is administered as an intravenous infusion within 1.5 to 2 hours.

In some embodiments, the anti-CD19 antibody is administered as an intravenous infusion at a dose of 12 mg/kg within 2 hours for the first one, two and three administrations and wherein after such first one, two and three administrations the anti-CD19 antibody is administered at a dose of at least 24 mg/kg (for example 24 mg/kg or 30 mg/kg) and wherein for the first one, two and three administrations said increased dose of at least 24 mg/kg (for example 24 mg/kg or 30 mg/kg) is administered as an intravenous infusion within 4 hours and wherein for all subsequent administrations said increased dose of at least 24 mg/kg (for example 24 mg/kg or 30 mg/kg) is administered as an intravenous infusion within 2 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 1.5 to 2.5 hours;
on day 15 of the first 28 days treatment cycle and on days 1 and 15 of the second and any subsequent 28 days treatment cycle said anti-CD19 antibody is administered at a dose of at least 24 mg/kg (for example 24 mg/kg or 30 mg/kg) and within 3 to 4.5 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 1.5 to 2.5 hours;
on day 15 of the first 28 days treatment cycle and on days 1 and 15 of the second and any subsequent 28 days treatment cycle said anti-CD19 antibody is administered at a dose of at least 24 mg/kg (for example 24 mg/kg or 30 mg/kg) and within 4 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 1.5 to 2.5 hours;
on day 15 of the first 28 days treatment cycle and on days 1 and 15 of the second and any subsequent 28 days treatment cycle said anti-CD19 antibody is administered at a dose of at least 24 mg/kg (for example 24 mg/kg or 30 mg/kg) and within 1.5 to 2.5 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 1.5 to 2.5 hours;
on day 15 of the first 28 days treatment cycle and on days 1 and 15 of the second and any subsequent 28 days treatment cycle said anti-CD19 antibody is administered at a dose of at least 24 mg/kg (for example 24 mg/kg or 30 mg/kg) and within 2 to 2.5 or within 3 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 1.5 to 2.5 hours;
on day 15 of the first 28 days treatment cycle and on days 1 and 15 of the second and any subsequent 28 days treatment cycle said anti-CD19 antibody is administered at a dose of at least 24 mg/kg (for example 24 mg/kg or 30 mg/kg) and within 2 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 2 hours;
on day 15 of the first 28 days treatment cycle and on days 1 and 15 of the second and the third 28 days treatment cycles said anti-CD19 antibody is administered at a dose of at least 24 mg/kg (for example 24 mg/kg or 30 mg/kg) and within 4 hours and
on day 1 of the fourth 28 days treatment cycle and on day 1 of any subsequent 28 days treatment cycle said anti-CD19 antibody is administered at a dose of at least 24 mg/kg (for example 24 mg/kg or 30 mg/kg) and within 2 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 1.5 to 2.5 hours;
on day 15 of the first 28 days treatment cycle and on days 1 and 15 of the second and the third 28 days treatment cycles said anti-CD19 antibody is administered at a dose of at least 24 mg/kg (for example 24 mg/kg or 30 mg/kg) and within 3 to 4.5 hours and
on day 1 of the fourth 28 days treatment cycle and on day 1 of any subsequent 28 days treatment cycle said anti-CD19 antibody is administered at a dose of at least 24 mg/kg (for example 24 mg/kg or 30 mg/kg) and within 1.5 to 2 hours.

In some embodiments, the present disclosure relates to a method of treating a cancer in a human subject in need thereof, the method comprising administering to the human subject an anti-CD19 antibody, wherein said anti-CD19 antibody is administered as an intravenous infusion according to the following schedule:
On days 1, 4 and 8 of a first 28 day treatment cycle said anti-CD19 antibody is administered at a dose of 12 mg/kg and within 2 hours;
on day 15 of the first 28 days treatment cycle and on days 1 and 15 of the second and the third 28 days treatment cycles said anti-CD19 antibody is administered at a dose of at least 24 mg/kg (for example 24 mg/kg or 30 mg/kg) and within 4 hours and
on day 1 of the fourth 28 days treatment cycle and on day 1 of any subsequent 28 days treatment cycle said anti-CD19 antibody is administered at a dose of at least 24 mg/kg (for example 24 mg/kg or 30 mg/kg) and within 2 hours.

In some embodiments, the anti-CD19 antibody is administered at a dose of 12 mg/kg on day 1, day 4 and day 8 from the start of treatment and from day 15 the anti-CD19 antibody is administered as an intravenous infusion at a dose of at least 24 mg/kg (for example 24 mg/kg or 30 mg/kg) and within 4 hours. In some embodiments, the anti-CD19 antibody is administered at a dose in the range of between 24 mg/kg to 30 mg/kg once a week, once every two weeks or once every four weeks.

In some embodiments, the anti-CD19 antibody is administered at a dose of 12 mg/kg on day 1, day 4 and day 8 from the start of treatment and from day 15 the anti-CD19 antibody is administered as an intravenous infusion at a dose of at least 24 mg/kg (for example 24 mg/kg or 30 mg/kg) and within 1.5 to 2.5 hours, within 1.5 to 2 hours, within 2 hours, within 2 to 2.5 hours, within 2 to 3 hours or within 3 hours. In some embodiments, the anti-CD19 antibody is administered at a dose in the range of between 24 mg/kg to 30 mg/kg once a week, once every two weeks or once every four weeks.

In some embodiments, the anti-CD19 antibody is administered at a dose of at least 24 mg/kg wherein the anti-CD19 antibody is administered as an intravenous infusion and wherein said intravenous infusion is administered within 1.5 to 2.5 hours and wherein the intravenous infusion is administered with an infusion rate of 70 mL/h for the first 30 minutes. In some other embodiments, the anti-CD19 antibody is administered at a dose of at least 24 mg/kg wherein the anti-CD19 antibody is administered as an intravenous infusion and wherein said intravenous infusion is administered within 1.5 to 2 hours and wherein the intravenous infusion is administered with an infusion rate of 70 mL/h for the first 30 minutes. In some other embodiments, the anti-CD19 antibody is administered at a dose of at least 24 mg/kg wherein the anti-CD19 antibody is administered as an intravenous infusion and wherein said intravenous infusion is administered within 2 hours and wherein the intravenous infusion is administered with an infusion rate of 70 mL/h for the first 30 minutes.

In some embodiments, the anti-CD19 antibody is administered at a dose of at least 24 mg/kg wherein the anti-CD19 antibody is administered as an intravenous infusion and wherein said intravenous infusion is administered within 3 to 4.5 hours and wherein the intravenous infusion is administered with an infusion rate of at least 30 mL/h for the first 30 minutes. In some other embodiments, the anti-CD19 antibody is administered at a dose of at least 24 mg/kg wherein the anti-CD19 antibody is administered as an intravenous infusion and wherein said intravenous infusion is administered within 3 to 4 hours and wherein the intravenous infusion is administered with an infusion rate of at least 30 mL/h for the first 30 minutes. In some other embodiments, the anti-CD19 antibody is administered at a dose of at least 24 mg/kg wherein the anti-CD19 antibody is administered as an intravenous infusion and wherein said intravenous infusion is administered within 3.5 to 4 hours and wherein the intravenous infusion is administered with an infusion rate of at least 30 mL/h for the first 30 minutes. In some other embodiments, the anti-CD19 antibody is administered at a dose of at least 24 mg/kg wherein the anti-CD19 antibody is administered as an intravenous infusion and wherein said intravenous infusion is administered within 3.5 to 4.5 hours and wherein the intravenous infusion is administered with an infusion rate of at least 30 mL/h for the first 30 minutes. In some other embodiments, the anti-CD19 antibody is administered at a dose of at least 24 mg/kg wherein the anti-CD19 antibody is administered as an intravenous infusion and wherein said intravenous infusion is administered within 4 hours and wherein the intravenous infusion is administered with an infusion rate of at least 30 mL/h for the first 30 minutes.

In some embodiments, the anti-CD19 antibody is administered in multiple administrations comprising a first administration and one or more subsequent administrations at a dose of at least 24 mg/kg, wherein each of the first administration and one or more subsequent administrations of the anti-CD19 antibody is administered as an intravenous infusion, and wherein said first intravenous infusion is administered within 1.5 to 2.5 hours with an infusion rate of 70 mL/h for the first 30 minutes. In some other embodiments, the first intravenous infusion is administered within 1.5 to 2 hours with an infusion rate of 70 mL/h for the first 30 minutes. In some other embodiments, the first intravenous infusion is administered within 2 hours with an infusion rate of 70 mL/h for the first 30 minutes. In some embodiments, the one or more subsequent intravenous infusions are administered within 1.5 to 2.5 hours, within 1.5 to 2 hours, or within 2 hours. In other embodiments, the anti-CD19 antibody is administered in multiple administrations comprising a first administration and one or more subsequent administrations at a dose of at least 24 mg/kg, wherein each of the first administration and one or more subsequent administrations of the anti-CD19 antibody is administered as an intravenous infusion and wherein said first intravenous infusion is administered within 1.5 to 2.5 hours with an infusion rate of 70 mL/h for the first 30 minutes and the one or more subsequent intravenous infusions are administered within 1.5 to 2 hours or within 2 hours.

In some embodiments, the anti-CD19 antibody is administered in multiple administrations comprising a first administration and one or more subsequent administrations at a dose of at least 24 mg/kg wherein each of the first administration and one or more subsequent administrations of the anti-CD19 antibody is administered as an intravenous infusion and wherein said first intravenous infusion is administered within 3 to 4.5 hours with an infusion rate of at least 30 mL/h for the first 30 minutes. In some other embodiments, the first intravenous infusion is administered within 3 to 4 hours with an infusion rate of at least 30 mL/h for the first 30 minutes. In some other embodiments, the first intravenous infusion is administered within 3.5 to 4 hours with an infusion rate of at least 30 mL/h for the first 30 minutes. In some other embodiments, the first intravenous infusion is administered within 3.5 to 4.5 hours with an infusion rate of at least 30 mL/h for the first 30 minutes. In some other embodiments, the first intravenous infusion is administered within 4 hours with an infusion rate of at least 30 mL/h for the first 30 minutes. In some embodiments, the one or more subsequent intravenous infusions are administered within 3 to 4.5 hours, within 3 to 4 hours, within 3.5 to 4 hours, within 3.5 to 4. 5 hours, or within 4 hours.

In some embodiments, the anti-CD19 antibody is administered at a dose of 24 mg/kg or 30 mg/kg wherein the anti-CD19 antibody is administered as an intravenous infusion and wherein said intravenous infusion is administered within 1.5 to 2.5 hours. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 1.5 to 2 hours. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 2 hours.

In some embodiments, the anti-CD19 antibody is administered at a dose of 24 mg/kg or 30 mg/kg wherein the anti-CD19 antibody is administered as an intravenous infusion and wherein said intravenous infusion is administered within 3 to 4.5 hours. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 3 to 4 hours. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 3.5 to 4 hours. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 3.5 to 4.5 hours. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 4 hours.

In some embodiments, the anti-CD19 antibody is administered at a dose of 24 mg/kg or 30 mg/kg wherein the anti-CD19 antibody is administered as an intravenous infusion and wherein said intravenous infusion is administered within 1.5 to 2.5 hours and wherein the intravenous infusion is administered with an infusion rate of 70 mL/h for the first 30 minutes. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 1.5 to 2 hours and wherein the intravenous infusion is administered with an infusion rate of 70 mL/h for the first 30 minutes. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 2 hours and wherein the intravenous infusion is administered with an infusion rate of 70 mL/h for the first 30 minutes.

In some embodiments, the anti-CD19 antibody is administered at a dose of 24 mg/kg or 30 mg/kg wherein the anti-CD19 antibody is administered as an intravenous infusion and wherein said intravenous infusion is administered within 3 to 4.5 hours and wherein the intravenous infusion is administered with an infusion rate of at least 30 mL/h for the first 30 minutes. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 3 to 4 hours and wherein the intravenous infusion is administered with an infusion rate of at least 30 mL/h for the first 30 minutes. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 3.5 to 4 hours and wherein the intravenous infusion is administered with an infusion rate of at least 30 mL/h for the first 30 minutes. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 3.5 to 4.5 hours and wherein the intravenous infusion is administered with an infusion rate of at least 30 mL/h for the first 30 minutes. In some other embodiments, the anti-CD19 antibody is administered at a dose of at least 24 mg/kg (for example 24 mg/kg or 30 mg/kg) wherein the anti-CD19 antibody is administered as an intravenous infusion and wherein said intravenous infusion is administered within 4 hours and wherein the intravenous infusion is administered with an infusion rate of at least 30 mL/h for the first 30 minutes.

In some embodiments, the anti-CD19 antibody is administered in multiple administrations comprising a first administration and one or more subsequent administrations at a dose of at least 24 mg/kg, for example 24 mg/kg or 30 mg/kg, wherein each of the first administration and one or more subsequent administrations of the anti-CD19 antibody is administered as an intravenous infusion and wherein said first intravenous infusion is administered within 1.5 to 2.5 hours with an infusion rate of 70 mL/h for the first 30 minutes. In some other embodiments, the first intravenous infusion is administered within 1.5 to 2 hours with an infusion rate of 70 mL/h for the first 30 minutes. In some other embodiments, the first intravenous infusion is administered within 2 hours with an infusion rate of 70 mL/h for the first 30 minutes. In some embodiments, the one or more subsequent intravenous infusions are administered within 1.5 to 2.5 hours, within 1.5 to 2 hours, or within 2 hours. In other embodiments, the anti-CD19 antibody is administered in multiple administrations comprising a first administration and one or more subsequent administrations at a dose of at least 24 mg/kg, for example 24 mg/kg or 30 mg/kg, wherein each of the first administration and one or more subsequent administrations of the anti-CD19 antibody is administered as an intravenous infusion and wherein said first intravenous infusion is administered within 1.5 to 2.5 hours with an infusion rate of 70 mL/h for the first 30 minutes and the one or more subsequent intravenous infusions are administered within 1.5 to 2 hours or within 2 hours.

In some embodiments, the anti-CD19 antibody is administered in multiple administrations comprising a first administration and one or more subsequent administrations at a dose of at least 24 mg/kg, for example 24 mg/kg or 30 mg/kg, wherein each of the first administration and one or more subsequent administrations of the anti-CD19 antibody is administered as an intravenous infusion and wherein said first intravenous infusion is administered within 3 to 4.5 hours with an infusion rate of at least 30 mL/h for the first 30 minutes. In some other embodiments, the first intravenous infusion is administered within 3 to 4 hours with an infusion rate of at least 30 mL/h for the first 30 minutes. In some other embodiments, the first intravenous infusion is administered within 3.5 to 4 hours with an infusion rate of at least 30 mL/h for the first 30 minutes. In some other embodiments, the first intravenous infusion is administered within 3.5 to 4.5 hours with an infusion rate of at least 30 mL/h for the first 30 minutes. In some other embodiments, the first intravenous infusion is administered within 4 hours with an infusion rate of at least 30 mL/h for the first 30 minutes. In some embodiments, the one or more subsequent intravenous infusions are administered within 3 to 4.5 hours, within 3 to 4 hours, within 3.5 to 4 hours, within 3.5 to 4. 5 hours, or within 4 hours.

In some embodiments, the anti-CD19 antibody is administered on Day 1 of a treatment cycle. In some embodiments, the treatment cycle is 28 days.

In some embodiments, the anti-CD19 antibody is administered at a dose of at least 24 mg/kg. In some embodiments, the anti-CD19 antibody is administered at a dose of at least 24 mg/kg once a week, once every two weeks or once every four weeks.

In some embodiments, the anti-CD19 antibody is administered at a dose of at least 24 mg/kg, wherein the anti-CD19 antibody is administered at a dose of 12 mg/kg prior to an increase to the dose of at least 24 mg/kg. In some embodiments, the anti-CD19 antibody is administered at a dose of 12 mg/kg for the first one, two or three administrations and wherein after such first one, two or three administrations the anti-CD19 antibody is administered at a dose of at least 24 mg/kg. In some embodiments, the anti-CD19 antibody is administered at a dose of at least 24 mg/kg once a week, once every two weeks or once every four weeks.

In some embodiments, the anti-CD19 antibody is administered at a dose of 12 mg/kg on day 1, day 4 and day 8 from the start of treatment and from day 15 the anti-CD19 antibody is administered at a dose of at least 24 mg/kg. In some embodiments, the anti-CD19 antibody is administered at a dose of at least 24 mg/kg once a week, once every two weeks or once every four weeks.

In some embodiments, the anti-CD19 antibody is administered in 28-day cycles, wherein on: a) Days 1, 4 and 9 of the first cycle, a dose of 12 mg/kg is administered and on day 15 of the first cycle a dose of at least 24 mg/kg is administered; b) Days 1 and 15 of cycles 2-3, a dose of at least 24 mg/kg is administered; and c) Day 1 of further subsequent cycles, a dose of at least 24 mg/kg is administered.

In some embodiments, the anti-CD19 antibody is administered at a dose in the range of between 24 mg/kg to 30 mg/kg. In some embodiments, the anti-CD19 antibody is administered at a dose in the range of between 24 mg/kg to 30 mg/kg. In some embodiments, the anti-CD19 antibody is administered at a dose in the range of between 24 mg/kg to 30 mg/kg once a week, once every two weeks or once every four weeks.

In some embodiments, the anti-CD19 antibody is administered at a dose in the range of between 24 mg/kg to 30 mg/kg, wherein the anti-CD19 antibody is administered at a dose of 12 mg/kg prior to an increase to the dose in the range of between 24 mg/kg to 30 mg/kg. In some embodiments, the anti-CD19 antibody is administered at a dose of 12 mg/kg for the first one, two or three administration and wherein after such first one, two or three administrations the anti-CD19 antibody is administered at a dose in the range of between 24 mg/kg to 30 mg/kg. In some embodiments, the anti-CD19 antibody is administered at a dose in the range of between 24 mg/kg to 30 mg/kg once a week, once every two weeks or once every four weeks.

In some embodiments, the anti-CD19 antibody is administered at a dose of 12 mg/kg on day 1, day 4 and day 8 from the start of treatment and from day 15 the anti-CD19 antibody is administered at a dose in the range of between 24 mg/kg to 30 mg/kg. In some embodiments, the anti-CD19 antibody is administered at a dose in the range of between 24 mg/kg to 30 mg/kg once a week, once every two weeks or once every four weeks.

In some embodiments, the anti-CD19 antibody is administered in 28-day cycles, wherein on: a) Days 1, 4 and 9 of the first cycle, a dose of 12 mg/kg is administered and on day 15 of the first cycle a dose in the range of between 24 mg/kg to 30 mg/kg is administered; b) Days 1 and 15 of cycles 2-3, a dose in the range of between 24 mg/kg to 30 mg/kg is administered; and c) Days 1 of further subsequent cycles, a dose in the range of between 24 mg/kg to 30 mg/kg is administered.

In some embodiments, the anti-CD19 antibody is administered at a dose of 24 mg/kg. In some embodiments, the anti-CD19 antibody is administered at a dose of at 24 mg/kg once every two weeks.

In some embodiments, the anti-CD19 antibody is administered at a dose of 24 mg/kg, wherein the anti-CD19 antibody is administered at a dose of 12 mg/kg prior to an increase to the dose of 24 mg/kg. In some embodiments, the anti-CD19 antibody is administered at a dose of 12 mg/kg for the first one, two or three administrations and wherein after such first one, two or three administrations the anti-CD19 antibody is administered at a dose of 24 mg/kg. In some embodiments, the anti-CD19 antibody is administered at a dose of 24 mg/kg once a week, once every two weeks or once every four weeks.

In some embodiments, the anti-CD19 antibody is administered at a dose of 12 mg/kg on day 1, day 4 and day 8 from the start of treatment and from day 15 the anti-CD19 antibody is administered at a dose of 24 mg/kg. In some embodiments, the anti-CD19 antibody is administered at a dose of 24 mg/kg once a week, once every two weeks or once every four weeks.

In some embodiments, the anti-CD19 antibody is administered in 28-day cycles, wherein on: a) Days 1, 4 and 9 of the first cycle, a dose of 12 mg/kg is administered and on day 15 of the first cycle a dose of 24 mg/kg is administered; b) Days 1 and 15 of cycles 2-3, a dose of 24 mg/kg is administered; and c) Day 1 of further subsequent cycles, a dose of 24 mg/kg is administered.

In some embodiments, the anti-CD19 antibody is administered at a dose of 30 mg/kg. In some embodiments, the anti-CD19 antibody is administered at a dose of 30 mg/kg once every two weeks.

In some embodiments, the anti-CD19 antibody is administered at a dose of 30 mg/kg, wherein the anti-CD19 antibody is administered at a dose of 12 mg/kg prior to an increase to the dose of 30 mg/kg. In some embodiments, the anti-CD19 antibody is administered at a dose of 12 mg/kg for the first one, two or three administrations and wherein after such first one, two or three administrations the anti-CD19 antibody is administered at a dose of 30 mg/kg. In some embodiments, the anti-CD19 antibody is administered at a dose of 30 mg/kg once a week, once every two weeks or once every four weeks.

In some embodiments, the anti-CD19 antibody is administered at a dose of 30 mg/kg, wherein the anti-CD19 antibody is administered at a dose of 12 mg/kg prior to an increase to a dose of 24 mg/kg and prior to an increase to the dose of 30 mg/kg. In some embodiments, the anti-CD19 antibody is administered at a dose of 12 mg/kg for the first one, two or three administrations and wherein after such first one, two or three administrations the anti-CD19 antibody is administered at a dose of 24 mg/kg and wherein after the administration at a dose of 24 mg/kg the anti-CD19 antibody is administered at a dose of 30 mg/kg. In some embodiments, the anti-CD19 antibody is administered at a dose of 30 mg/kg once a week, once every two weeks or once every four weeks.

In some embodiments, the anti-CD19 antibody is administered at a dose of 12 mg/kg on day 1, day 4 and day 8 from the start of treatment and from day 15 the anti-CD19 antibody is administered at a dose of 30 mg/kg. In some embodiments, the anti-CD19 antibody is administered at a dose of 30 mg/kg once a week, once every two weeks or once every four weeks.

In some embodiments, the anti-CD19 antibody is administered in 28-day cycles, wherein on: a) Days 1, 4 and 9 of the first cycle, a dose of 12 mg/kg is administered and on day 15 of the first cycle a dose of 30 mg/kg is administered; b) Days 1 and 15 of cycles 2-3, a dose of 30 mg/kg is administered; and c) Day 1 of further subsequent cycles, a dose of 30 mg/kg is administered.

In some embodiments, the anti-CD19 antibody is administered in combination with lenalidomide. In certain aspects, lenalidomide is administered orally. In certain aspects, lenalidomide is administered daily on days 1-21 of repeated 28-day cycles. In certain aspects, lenalidomide is administered daily on days 1-21 of up to 12 repeated 28-day cycles. In certain aspects, the dose of lenalidomide is at least 20 mg daily. In certain aspects, the dose of lenalidomide is 25 mg daily.

In some embodiments, the anti-CD19 antibody is administered in combination with lenalidomide wherein lenalidomide is administered orally at a dose of 25 mg on days 1-21 of repeated 28-day cycles. In some embodiments, the anti-CD19 antibody is administered in combination with lenalidomide wherein lenalidomide is administered orally at a dose of 25 mg and daily on days 1-21 of 12 repeated 28-day cycles.

### DESCRIPTION OF DRAWINGS

FIG. 1 & FIG. 2: Boxplots of Model-predicted PK Parameters Based on 2000 Randomly Generated Patients Dosed According to the L-MIND, the 12/24 mg/kg (Cohort 1) and the 12/30 mg/kg (Cohort 2) Dosing Regimen.

FIG. 3: Overlay of the Model-predicted Median of the Concentration-time Profiles of Tafasitamab Dosed According to the L MIND and the 12/30 mg/kg Dosing Regimen.

### DEFINITIONS AND DETAILED DESCRIPTION

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12. The term also encompasses naturally occurring variants of CD19, e.g., splice variants, allelic variants, and isoforms.

In one embodiment human CD19 has the amino acid sequence of:

(SEQ ID NO: 13)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCLPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKRKRMTDPTRRFFKVTPPPGSG
PQNQYGNVLSLPTPTSGLGRAQRWAAGLGGTAPSYGNPSSDVQADGALG
SRSPPGVGPEEEEGEGYEEPDSEEDSEFYENDSNLGQDQLSQDGSGYEN
PEDEPLGPEDEDSFSNAESYENEDEELTQPVARTMDFLSPHGSAWDPSR
EATSLGSQSYEDMRGILYAAPQLRSIRGQPGPNHEEDADSYENMDNPDG
PDPAWGGGGRMGTWSTR.

“MOR208” and “XmAb 5574” and “tafasitamab” are used as synonyms for the anti-CD19 antibody according to Table A. Table A provides the amino acid sequences of MOR208/tafasitamab. The MOR208 antibody is described in U.S. Pat. No. 8,524,867, which is incorporated by reference in its entirety (in U.S. Pat. No. 8,524,867, the full heavy chain of MOR208 is SEQ ID NO:87 and the full light chain of MOR208 is SEQ ID NO:106).

“Fc region” means the constant region of an antibody, which in humans may be of the IgG1, 2, 3, 4 subclass or others. The sequences of human Fc regions are available at the IMGT website.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses polyclonal antibodies, monoclonal antibodies, antibody fragments (such as Fab, Fab′, F(ab′)2, and Fv fragments), single chain Fv (scFv) mutants, multispecific antibodies such as bispecific antibodies for example generated from at least two intact antibodies, chimeric antibodies, humanized antibodies, human antibodies, fusion proteins comprising an antigen determination portion of an antibody, and any other modified immunoglobulin molecule comprising an antigen recognition site. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The different classes of immunoglobulins have different and well-known subunit structures and three-dimensional configurations. Antibodies can be naked or conjugated to other molecules such as toxins, radioisotopes, etc., either directly or through one or more linkers.

The term “anti-CD19 antibody” or “an antibody that binds to CD19” refers to an antibody that is capable of binding CD19 with sufficient affinity such that the antibody is useful as a diagnostic and/or therapeutic agent in targeting CD19.

A “monoclonal antibody” refers to a homogeneous or substantially homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

As used herein, the term “effective amount” or “therapeutically effective amount” refers to an amount of a compound, or combination of one or more compounds that, when administered (either sequentially or simultaneously) elicits the desired biological or medicinal response, e.g., either destroys, slows or arrests the growth of the target cancer cells, slows or arrests the progression of the cancer in a patient and/or delays, eliminates, reduces or otherwise ameliorates one or more symptoms of cancer in a patient. The therapeutically effective amount may vary depending upon the intended application, or the patient and disease condition being treated, and can depend on factors such as, e.g., the weight and age of the patient, the severity of the disease condition, the manner of administration and the like, which may readily be determined by one skilled in the art. The term “effective amount” or “therapeutically effective amount” also applies to an amount, such as one or more doses, that will induce a particular response in target cells, e.g., reduction of platelet adhesion and/or cell migration.

“Administered” or “administration” includes but is not limited to delivery by an injectable form, such as, for example, an intravenous, intramuscular, intradermal or subcutaneous route, or by a mucosal route, for example, as a nasal spray or aerosol for inhalation or as an ingestible solution, capsule or tablet.

Anti-CD19 Antibodies

CD19 is broadly and homogeneously expressed across different B-cell derived blood cancers. CD19 is able to enhance B-cell receptor signaling, which is important for B-cell survival, and is therefore a therapeutic target for drugs aimed at treating B cell-related lymphomas and leukemias.

Antibodies, such as tafasitamab, can be made, for example, by preparing and expressing synthetic genes that encode the recited amino acid sequences or by mutating human germline genes to provide a gene that encodes the recited amino acid sequences. Moreover, this antibody and other anti-CD19 antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064. Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., a human IgG1 constant region is mutated to include the S239D and/or I332E substitutions. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-CD19 antibodies can be in the form of full length antibodies, or in the form of low molecular weight forms (e.g., biologically active antibody fragments or minibodies) of the anti-CD19 antibodies, e.g., Fab, Fab′, F(ab′)2, Fv, Fd, dAb, scFv, and sc(Fv)2. Other anti-CD19 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-CD19 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 30 10%, 5% or 1%. Also provided are compositions comprising an anti-CD19 antibody or antigen binding fragment thereof comprising at least one deamidation site, wherein the pH of the composition is from about 5.0 to about 6.5, such that, e.g., at least about 90% of the anti-CD19 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising an antiCD19 antibody or antigen-binding fragment thereof, means the presence of both the desired antiCD19 antibody or antigen-binding fragment thereof and one or more acidic variants thereof. The acidic variants may comprise predominantly deamidated anti-CD19 antibody, with minor amounts of other acidic variant(s).

In certain embodiments, the binding affinity (KD), on-rate (KD on) and/or off-rate (KD off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

Bispecific Antibodies

In certain embodiments, an anti-CD19 antibody or antigen-binding fragment thereof described herein is present in a bispecific antibody. Exemplary bispecific antibodies may bind to two different epitopes of the CD19 protein. Other such antibodies may combine a CD19 binding site with a binding site for another protein. Bispecific antibodies can be prepared as full length antibodies or low molecular weight forms thereof (e.g., F(ab′) 2 bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

Multivalent Antibodies

In certain embodiments, an anti-CD19 antibody or antigen-binding fragment thereof described herein is present in a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is a polypeptide chain of an Fc region, X1 and X2 represent an amino acid or peptide spacer, and n is 0 or 1.

Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., 90Y, 1311) fluorescent substances, luminescent substances, haptens, enzymes, metal chelates, drugs, and toxins (e.g., calcheamicin, Pseudomonas exotoxin A, ricin (e.g. deglycosylated ricin A chain) and auristatins such as auristatin E and auristatin F).

In one embodiment, to improve the cytotoxic actions of anti-CD19 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CD19 antibody or antigen-binding fragment thereof are modified with a moiety that improves its stabilization and/or retention in circulation, e.g., in blood, serum, or other tissues, e.g., by at least 1.5, 2, 5, 10, or 50 fold. For example, the anti-CD19 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CD19 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides. The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 15 5,156,840).

Methods of Producing Antibodies

Antibodies may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5 [symbol] , HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell. If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1 [symbol] promoter (Mizushima et al, Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CD19 antibody (e.g., tafasitamab) is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium. Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS and Sepharose FF (GE Healthcare Biosciences). The present disclosure also includes antibodies that are highly purified using these purification methods.

Antibody Pharmaceutical Compositions and Administration

An anti-CD19 antibody or antigen-binding fragment thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The anti-CD19 antibody or antigen-binding fragment thereof can be administered to a subject, e.g., a subject in need thereof, for example, a human subject, by a variety of methods.

For many applications, the route of administration is one of: intravenous injection or infusion (IV), subcutaneous injection (SC), intraperitoneally (IP), or intramuscular injection. It may also be possible to use intra-articular delivery. Other modes of parenteral administration can also be used. In some cases, administration can be oral. The route and/or mode of administration of the antibody or antigen-binding fragment thereof can also be tailored for the individual case, e.g., by monitoring the subject, e.g., using tomographic imaging, e.g., to visualize a tumor.

A pharmaceutical composition may include a “therapeutically effective amount” of an anti-CD19 antibody or antigen-binding fragment thereof described herein. Such effective amounts can be determined based on the effect of the administered agent, or the combinatorial effect of agents if more than one agent is used, within the bounds of the doses and dosing regimens disclosed herein. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In certain embodiments, the anti-CD19 antibody replacing tafasitamab is a human, humanized, or chimeric antibody. In another embodiment of the present disclosure the anti-CD19 antibody replacing tafasitamab is of the IgG isotype. In another embodiment the antibody replacing tafasitamab is IgG1, IgG2, or IgG1/IgG2 chimeric. In another embodiment of the present disclosure the isotype of the anti-CD19 antibody replacing tafasitamab is engineered to enhance antibody-dependent cell-mediated cytotoxicity. In another embodiment the heavy chain constant region of the anti-CD19 antibody replacing tafasitamab comprises amino acids 239D and 332E, wherein the Fc numbering is according to the EU index as in Kabat. In another embodiment the anti-CD19 antibody replacing tafasitamab is IgG1, IgG2 or IgG1/IgG2, and the chimeric heavy chain constant region of the anti-CD19 antibody comprises amino acids 239D and 332E, wherein the Fc numbering is according to the EU index as in Kabat.

TABLE A
Tafasitamab (MOR208)

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSS |
| VL | SEQ ID NO: 8 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIK |
| Heavy chain constant | SEQ ID NO: 9 | ASTKGPSVFPLAPSSKSTSGGTAALGCLVKDY |
| domain | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPDVFLFPP |
| | | KPKDTLMISRTPEVTCVVVDVSHEDPEVQFNW |
| | | YVDGVEVHNAKTKPREEQFNSTFRVVSVLTVV |
| | | HQDWLNGKEYKCKVSNKALPAPEEKTISKTKG |
| | | QPREPQVYTLPPSREEMTKNQVSLTCLVKGFY |
| | | PSDIAVEWESNGQPENNYKTTPPMLDSDGSF |
| | | FLYSKLTVDKSRWQQGNVFSCSVMHEALHNH |
| | | YTQKSLSLSPGK |
| Light chain constant | SEQ ID NO: 10 | RTVAAPSVFIFPPSDEQLKSGTASVVCLLNNFY |
| domain | | PREAKVQWKVDNALQSGNSQESVTEQDSKD |
| | | STYSLSSTLTLSKADYEKHKVYACEVTHQGLS |
| | | SPVTKSFNRGEC |
| Full Heavy chain | SEQ ID NO: 11 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light chain | SEQ ID NO: 12 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIKRTVAAPSVFIFPPSDEQ |
| | | LKSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |

### Examples

The study: A Phase 1b/2, Open-Label, Multicenter Study to Evaluate the Safety and Pharmacokinetics of a Modified Tafasitamab IV Dosing Regimen Combined with Lenalidomide (LEN) in Patients with Relapsed or Refractory Diffuse Large B-Cell Lymphoma (R/R DLBCL) (MINDway).

To evaluate a tafasitamab dosing regimen with approximately 50% fewer iv. infusions than the currently approved drug regimen, the following modifications to the currently approved dosing regimen were defined:
The dosing frequency is planned to be reduced from once weekly (QW) to once every two weeks (Q2W) from Cycle 1 Day 15 (C1D15) onwards and from Q2W to once every 4 weeks (Q4W) from C4D1 onwards
The tafasitamab administration at the previous dose level of 12 mg/kg is planned to be maintained for the first three infusions (i.e., Days 1, 4 and 8); the tafasitamab dose level will be increased from C1D15 onwards

The stepwise dose increase is implemented as a risk minimization measure to limit potential AEs occurring shortly aftertreatment initiation (e.g., IRRs or tumor lysis syndrome [TLS]). Taking this new dosing schedule into account (see Table 2), a tafasitamab POP-PK model was used to identify new dose levels leading to similar tafasitamab trough levels as observed in the pivotal clinical trial L-MIND in order to maintain the previously established exposure/efficacy relationship. 24 mg/kg and 30 mg/kg administered according to the dosing schedule outlined in Table 2 were identified as potential new dose levels tested in the current trial.

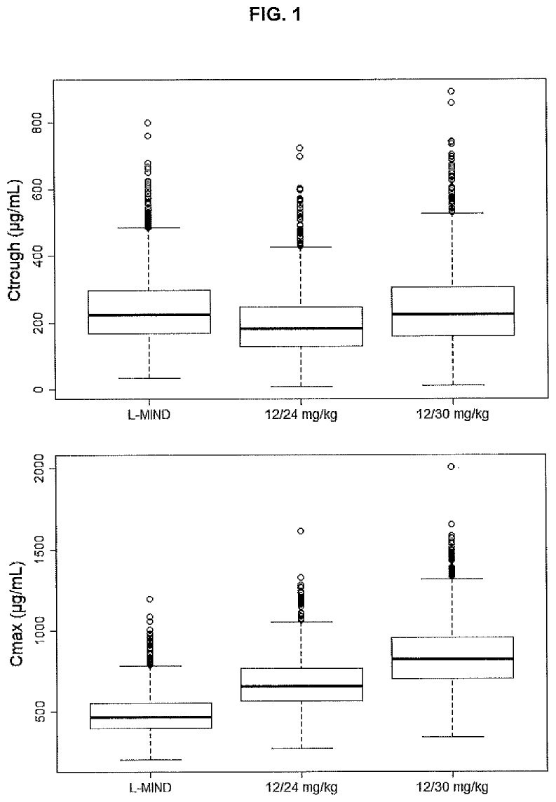

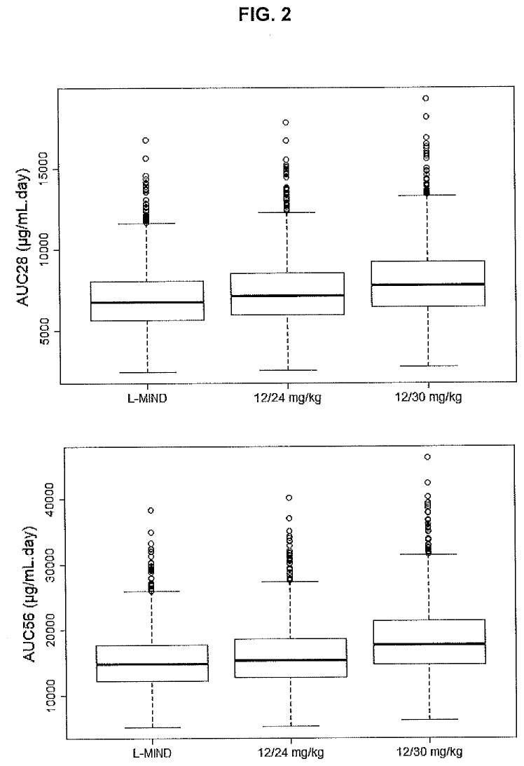

Box plots comparing the predicted minimum concentration (C_{trough}) and maximum concentration (Cₘₐₓ) values at the end of Cycle 3 as well as the predicted area under the curve (AUC) levels after one and two treatment cycles (AUC28 and AUC56) between the L-MIND, the 12/24 mg/kg and the 12/30 mg/kg dosing schedules are shown in FIGS. 1 & 2. The model predictions show that tafasitamab doses of 30 mg/kg are required to achieve C_{trough} levels as previously observed in L-MIND. In contrast, C_{trough} levels for the 12/24 mg/kg dosing regimen are predicted to be lower than the L-MIND trough levels (reduction in geometric mean concentrations vs L-MIND: 2.9% for 12/30 mg/kg vs 21.8% for 12/24 mg/kg).

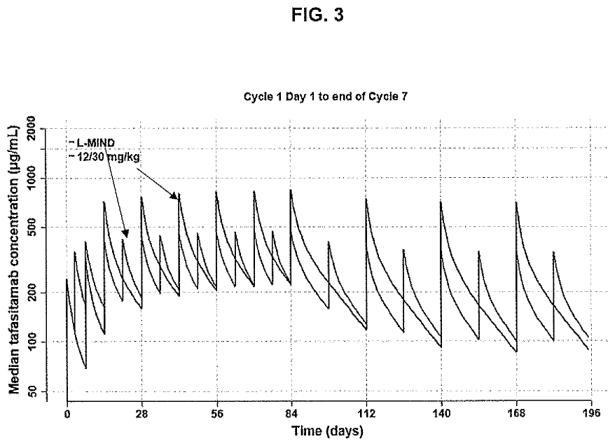

FIG. 3 shows a comparison between the median model-predicted tafasitamab concentrations over time between the L-MIND and the targeted 12/30 mg/kg dosing regimen and confirms that similar tafasitamab C_{trough} levels are achieved with the two different dosing schedules. Additional details on the simulations can be found in the MorphoSys report MOR208L050. In addition, the simulations were used to compare the expected exposure of the planned study.

MOR208C115 with previous non-clinical safety studies and clinical study:
In cynomolgus monkeys receiving QW doses of 100 mg/kg (i.e. at NOAEL), _{AUC0-144h} and Cₘₐₓ of tafasitamab at steady state are expected to be approximately 6-7-fold higher as compared to R/R DLBCL patients dosed according to the 12/24 mg/kg regimen and approximately 5-fold higher as compared to patients dosed according to the 12/30 mg/kg regimen
In comparison to clinical trial L-MIND, Cₘₐₓ concentrations are expected to be 1.4- and 1.7-fold higher for the 12/24 mg/kg and 12/30 mg/kg dosing respectively; in comparison to B-MIND, Cₘₐₓ concentrations are expected to be 1.2- and 1.4-fold higher for the 12/24 mg/kg and 12/30 mg/kg dosing respectively

In conclusion, the 12/30 mg/kg dosing regimen represents the targeted dosing regimen based on the expected similar C_{trough} levels as in L-MIND. However, in order to enable a stepwise evaluation of the safety of increasing tafasitamab Cₘₐₓ levels, enrollment of patients will start in Cohort 1 according to the 12/24 mg/kg dosing scheme. In case safety data support further dose escalation, the targeted 12/30 mg/kg dosing scheme will be investigated in Cohort 2.

Benefit/Risk Assessment

The risk assessment of tafasitamab and LEN is based on the data from nonclinical studies as well as on clinical experience from completed and ongoing clinical trials. Tafasitamab monotherapy was well tolerated in R/R B-cell lymphomas (BCLs) (Jurczak et al., 2018). Similarly, tafasitamab plus LEN showed a manageable safety profile in the L-MIND trial in R/R DLBCL (Salles et al., 2020) and received FDA approval in the US on Jul. 31, 2020.

In June 2021, European Medicines Agency's Committee for Medicinal Products for Human Use (CHMP) issued a positive opinion recommending the conditional marketing authorization of tafasitamab in combination with lenalidomide, followed by tafasitamab monotherapy, for the treatment of adult patients with relapsed or refractory diffuse large B-cell lymphoma (DLBCL) who are not eligible for autologous stem cell transplantation (ASCT).

More detailed information about the known and expected benefits and risks and reasonably expected AEs of tafasitamab is available in the current version of the IB.

### Risk Assessment

Tafasitamab is approved for use at a dose of 12 mg/kg. With the study dosing regimens as shown in Table 2 (combination of 12/24 mg/kg or 12/30 mg/kg) higher tafasitamab Cₘₐₓ levels than previously observed in L-MIND are expected. Based on the current non-clinical and clinical safety data, no correlation between the occurrence of AEs and exposure (including Cₘₐₓ) was observed. Nevertheless, the expected range of Cₘₐₓ values of the study dosing regimens is higher than and not covered by previous clinical data. In order to reduce the risk of potential AEs occurring shortly after treatment initiation such as IRRs and TLS, all patients will maintain a dose of 12 mg/kg at the initial three tafasitamab administrations on C1D1, C1D4 and C1D8 prior to dose increase to 24 mg/kg or 30 mg/kg. In addition, the infusion duration for the higher tafasitamab doses will be doubled, to keep similar tafasitamab infusion rates (in mg/h) as in L-MIND. An internal DSMC will monitor safety events during the study conduct and decide on dose change based on emerging safety, pharmacokinetic and pharmacodynamic data. Based on the clinical experience of tafasitamab in combination with LEN from L-MIND, the most common side effects (20%) of tafasitamab plus LEN were neutropenia, fatigue, anemia, diarrhea, thrombocytopenia, cough, pyrexia, peripheral edema, respiratory tract infection, and decreased appetite. Serious adverse events (SAEs) were reported in 6% of patients, included infections including pneumonia and febrile neutropenia.

At a first data cut-off on 10 Oct. 2022, two patients that were administered the 24 mg/kg dose in a doubled infusion duration (four hours) tolerated the infusions well and showed no obvious IRRs. One patient was administered a 24 mg/kg dose in an infusion duration of 2 hours and also tolerated the infusion without any obvious IRRs.

The modified dosing regimen (i.e., reducing the overall frequency of clinic visits by half) results in reduced patient burden and support long term treatment compliance. Furthermore, given the severity of disease, reduced hospital visits may result in less exposure to hospital infections in the already susceptible patient population.

The combination of tafasitamab plus LEN provides clinical benefit for patients with R/R DLBCL as demonstrated in the L-MIND trial. Based on the clinical efficacy data as of data cut-off of 30 Oct. 2020, for patients who had their DLBCL diagnosis confirmed by central pathology, combining tafasitamab plus LEN, showed an ORR of 53.5% (95% Cl: 41.3; 65.5) with a CR rate of 35.2% (95% Cl: 24.2; 47.5) and a median DoR of 43.9 months (95% Cl: 15.0; no response [NR]). Compared with monotherapy of tafasitamab and LEN, an ORR of >50% is considered highly clinically meaningful in the relapse-refractory setting of DLBCL not eligible to high dose chemotherapy and ASCT. Furthermore, the safety data from the L-MIND trial indicate that the addition of tafasitamab to LEN adds little additional toxicity, based on the characterized safety profile of LEN administered as monotherapy.

The new dosing regimen is expected to reduce the patient burden while achieving the same trough levels as in L-MIND. Furthermore, the same clinical efficacy rates are expected. Due to the expected widely overlapping Cₘₐₓ values (L-MIND vs. 12/24 mg/kg and 12/30 mg/kg predictions), no new types of safety events are expected.

Objectives, Endpoints, and Estimands

The following objectives and endpoints will be evaluated in the study, see Table 1:

TABLE 1
Objectives and Corresponding Endpoints

| Objectives | Endpoints |
|---|---|
| Primary | |
| To evaluate the safety and tolerability of | Incidence and severity of TEAEs |
| tafasitamab administered once every 2 weeks | |
| (Q2W)/once every 4 weeks (Q4W) in | |
| combination with lenalidomide in R/R DLBCL | |
| patients | |
| To determine a recommended dose for | |
| tafasitamab Q2W/Q4W administration in | |
| combination with lenalidomide in R/R DLBCL | |
| patients | |
| Secondary | |
| To evaluate the pharmacokinetic profile of | Tafasitamab serum concentrations after 3 |
| tafasitamab after Q2W/Q4W dosing in | (C_{trough} and Cₘₐₓ) and 12 (C_{trough}) treatment cycles |
| combination with lenalidomide | |
| To assess anti-tumor activity of tafasitamab | Best Objective Response Rate (ORR) by |
| after Q2W/Q4W dosing in combination with | Investigator |
| lenalidomide | assessment up to treatment Cycle 12 based on |
| | Cheson et al. (2007) |
| | Duration of Response (DoR) by Investigator |
| | assessment |
| | based on Cheson et al. (2007) |
| | Progression-Free Survival by Investigator |
| | assessment |
| | based on Cheson et al. (2007) |
| To assess the incidence of anti-drug | Number and percentage of patients developing |
| antibodies to tafasitamab | anti tafasitamab antibodies up to treatment |
| | Cycle 12 |

Abbreviations: Cₘₐₓ = maximum concentration; C_{trough} = minimum concentration; DLBCL = diffuse large B-cell lymphoma; NK = natural killer; Q2W = once every 2 weeks; Q4W = once every 4 weeks; R/R = relapsed/refractory; TEAEs = treatment-emergent adverse events.
Each treatment cycle is 28 days.

### Estimands

The primary clinical question of interest is: Given a proposed alternative treatment dosing regimen with tafasitamab at higher doses than previously investigated, what is the observed incidence and severity of treatment emergent adverse events (TEAEs) in R/R DLBCL patients who receive at least one dose at either 24 or 30 mg/kg? This would allow the assessment of the safety and the tolerability of the alternative treatment-dosing regimen. The primary estimand and its attributes are described in Section 4. Possible intercurrent events and strategies to capture them are described in Section 4.

### Study Design

MOR208C115 (MINDway) is an open-label, multicenter, phase 1b/2 study of tafasitamab combined with lenalidomide (LEN) to evaluate a modified tafasitamab dosing regimen in adult patients with R/R DLBCL. Overall, approximately 51 patients will be enrolled in the study. Patients will receive LEN in combination with tafasitamab in 28-day cycles. The modified tafasitamab dosing regimen will be investigated in a stepwise design with two sequential cohorts followed by an expansion cohort at the recommended dose level. Tafasitamab will be administered as intravenous infusion according to the following dosing schedule (Table 2).

TABLE 2
Dosing Schedule and Frequency

| | | LEN |
|---|---|---|
| Cohort | Tafasitamab¹ | (up to 12 cycles) |
| 1 | Cycle 1: 12 mg/kg on Days 1, 4 and 8 | Cycle 1-12: |
| | Cycle 1: 24 mg/kg on Day 15 | 25 mg on Days |
| | Cycles 2 and 3: 24 mg/kg on Days 1 | 1-21 of each cycle |
| | and 15 of each 28-day cycle | |
| | Cycle 4 and beyond: 24 mg/kg on | |
| | Day 1 of each 28-day cycle | |
| 2 | Cycle 1: 12 mg/kg on Days 1, 4 and 8 | |
| | Cycle 1: 30 mg/kg on Day 15 | |
| | Cycles 2 and 3: 30 mg/kg on Days 1 | |
| | and 15 of each 28-day cycle | |
| | Cycle 4 and beyond: 30 mg/kg on | |
| | Day 1 of each 28-day cycle | |
| Expansion | In the Expansion Cohort tafasitamab | |
| | will be administered according to the | |
| | selected dose regimen (24 mg/kg or | |
| | 30 mg/kg) for further | |
| | evaluation | |

¹Each treatment cycle is 28 days.

LEN (25 mg) will be administered for a maximum of 12 cycles or until disease progression, unacceptable toxicity, withdrawal, death or lost to follow up, whichever comes first. After cycle 12 or LEN discontinuation, patients will continue with tafasitamab monotherapy at the assigned dosing regimen until disease progression, unacceptable toxicity, withdrawal, death or lost to follow up, whichever comes first. No follow-up for overall survival will be performed in this study after end of treatment (EOT).

A Data and Safety Monitoring Committee (DSMC), consisting of Sponsor representatives and Investigators, will continuously monitor the study and can recommend to stop enrollment at any time based on emerging safety data. In addition, pre-defined DSMC meetings will take place when at least 6 patients have completed the 5-week (35 day) safety observation period in Cohort 1 and Cohort 2, respectively.

Details of specific responsibilities, composition, meeting formats and frequency of the DSMC are outlined in the DSMC Charter.

### Study Population

The study will enroll approximately 51 patients with histologically confirmed diagnosis of R/R DLBCL (as specified in inclusion criterion 3) based on the local pathology report.

All patients must meet below listed eligibility criteria to be enrolled in the study. Prospective approvals of deviations to eligibility criteria, also known as protocol waivers or exemptions, are not permitted.

### Inclusion Criteria

Patients are eligible to be included in the study only if all the following criteria apply:
1. Capable of giving signed informed consent: Regulatory, Ethical, and Trial Oversight Considerations, which includes compliance with the requirements and restrictions listed in the informed consent form (ICF) and in this protocol.
2. Patient must be 18-80 years of age (18-70 for Czech republic) at the time of signing the informed consent.
3. One of the following histologically confirmed diagnoses:
   DLBCL not otherwise specified (NOS)
   T cell/histiocyte-rich large B-cell lymphoma (THRLBCL)
   Epstein-Barr virus (EBV) positive DLBCL of the elderly (EBV-positive DLBCL)
   Grade 3b Follicular Lymphoma
   Composite lymphoma with a DLBCL component with a subsequent DLBCL
relapse, according to the Revised European American Lymphoma/World Health Organization (REAL/WHO) classification.

Additionally, patients with the evidence of histological transformation to DLBCL from an earlier diagnosis of low-grade lymphoma (i.e., an indolent pathology such as follicular lymphoma, marginal zone lymphoma, chronic lymphocytic leukemia) into DLBCL with a subsequent DLBCL relapse are also eligible.
4. Tumor tissue for retrospective central pathology review must be provided as an adjunct to participation in this study. If archival paraffin embedded tumor tissue acquired ≤3 years prior to screening is not available, a fresh tumor tissue sample from the patient should be obtained.
5. Patients must have:
a. Relapsed and/or refractory disease as defined in Appendix 3: Study Specific Definitions Appendix
b. At least one bidimensionally measurable disease site. The lesion must have a greatest transverse diameter of ≥1.5 cm and greatest perpendicular diameter of 1.0 cm at baseline. The lesion must be positive on positron emission tomography (PET) scan (for definition see Juweid et al., 2007)
c. Received at least one, but no more than three previous systemic regimens for the treatment of DLBCL and one therapy line must have included a cluster of differentiation-20 (CD20)-targeted therapy (e.g., rituximab [RTX])
d. An Eastern Cooperative Oncology Group (ECOG) performance status of 0 to 2.
6. Patients that are not eligible, or patients unwilling to undergo intensive salvage therapy including autologous stem cell transplantation (ASCT). The reason for a patient's ineligibility must meet one of the criteria described below and documented in the patient's source data:
a. Inadequate performance status (Karnofsky performance status ≤80%; see Karnofsky Performance Status Scale)
b. Disease not responsive to salvage chemotherapy. Responsiveness is defined as a tumor demonstrating either complete response (CR) or partial response (PR) to salvage chemotherapy
c. Inadequate major organ function (any of the below):
   i. symptomatic congestive heart failure
   ii. lung function-forced vital capacity (FVC), forced expiratory volume in 1 second (FEV-1), and corrected diffusion capacity of the lung for carbon monoxide (DLCO)≤60%
   iii. liver function-total serum bilirubin and transaminases >2× upper limit of normal (ULN)
d. History or evidence of significant co-morbid medical or psychiatric illness which would significantly compromise the patient's clinical care and chances of survival
e. Inability to collect adequate stem cell graft (e.g. <1-2×10⁶ CD34+ cells free of tumor contamination/kg recipient body weight)
7. Patients must meet the following laboratory criteria at screening:
a. Absolute neutrophil count (ANC)≥1.5×10⁹/L (unless secondary to bone marrow involvement by DLBCL as demonstrated by recent bone marrow aspiration and bone marrow biopsy)
b. Platelet count≥75×10⁹/L (unless secondary to bone marrow involvement by DLBCL as demonstrated by recent bone marrow aspiration and bone marrow biopsy)
c. Total serum bilirubin≤2.5×ULN unless secondary to Gilbert's syndrome or documented liver involvement by lymphoma. Patients with Gilbert's syndrome or documented liver involvement by lymphoma may be included if their total bilirubin is ≤5×ULN (see exclusion criterion 6g)
d. Alanine transaminase (ALT), aspartate transaminase (AST) and alkaline phosphatase (ALP)≤3×ULN or <5×ULN in cases of documented liver involvement
e. Serum creatinine CL must be ≥50 mL/minute either measured or calculated using a standard Cockcroft and Gault formula

### Exclusion Criteria

Patients are excluded from the study if any of the following criteria apply:
1. General provisions:
   a. Patients who are legally institutionalized, or patients under judicial protection
   b. Concurrent enrollment in another interventional clinical study
2. Patients who have:
   a. Any other histological type of lymphoma including primary mediastinal (thymic) large B-cell (PMBL) or Burkitt lymphoma
   b. Primary refractory DLBCL (see Appendix 3: Study Specific Definitions for definition)
   c. Known “double/triple hit” genetics (high grade B-cell lymphoma) characterized by simultaneous detection of MYC with BCL2 and/or BCL6 translocation(s) defined by fluorescence in situ hybridization. MYC, BCL2, BCL6 testing prior to study enrollment is not required
3. Patients who have, within 14 days prior to Day 1 dosing:
   a. Not discontinued CD20-targeted therapy, chemotherapy, radiotherapy, investigational anticancer therapy or other lymphoma-specific therapy
   b. Undergone major surgery (within 4 weeks) or suffered from significant traumatic injury
   c. Received live vaccines (see Appendix 7: Covid-19: Infection Prophylaxis and Vaccines)
   d. Required parenteral antimicrobial therapy for active, intercurrent infections
4. Patients who:
   a. Have, in the opinion of the investigator, not recovered sufficiently from the adverse toxic effects of prior therapies
   b. Were previously treated with CD19-targeted therapy or IMiDs® (e.g., thalidomide, LEN)
   c. Have a history of hypersensitivity to compounds of similar biological or chemical composition to tafasitamab, IMiDs® and/or the excipients contained in the study treatment formulations
   d. Have undergone ASCT within the period≤3 months prior to signing the ICF.
   Patients who have a more distant history of ASCT must exhibit full hematological recovery before enrolment into the study
   e. Have undergone previous allogenic stem cell transplantation
   f. Have a history of deep venous thrombosis/embolism, threatening thromboembolism or known thrombophilia or are at high risk for a thromboembolic event in the opinion of the investigator and who are not willing/able to take venous thromboembolism (VTE) prophylaxis during the entire treatment period
   g. Concurrently use other anticancer or experimental treatments
5. History of other malignancy that could affect compliance with the protocol or interpretation of results. Exceptions:
   a. Patients with any malignancy appropriately treated with curative intent and the malignancy has been in remission without treatment for >2 years prior to enrollment are eligible
   b. Patients with low-grade, early-stage prostate cancer (Gleason score 6 or below, Stage 1 or 2) with no requirement for therapy at any time prior to study are eligible
6. Patients with:
   a. Positive hepatitis B and/or C serology (see 12.8 Appendix 8: Hepatitis Virus Serology for details)
   b. Known seropositivity for or history of active viral infection with human immunodeficiency virus (HIV)
   c. Central nervous system (CNS) lymphoma involvement—present or past medical history
   d. History or evidence of clinically significant cardiovascular, CNS and/or other systemic disease that would in the investigator's opinion preclude participation in the study or compromise the patient's ability to give informed consent
   e. History or evidence of rare hereditary problems of galactose intolerance, the Lapp lactase deficiency or glucose-galactose malabsorption
   f. Gastrointestinal (GI) abnormalities (issue with absorption) including the inability to take oral medication
   g. History or evidence of severe hepatic impairment (total serum bilirubin>3 mg/dL), jaundice unless secondary to Gilbert's syndrome or documented liver involvement by lymphoma (see inclusion criterion 7c)
   h. History of hypersensitivity to any of the study treatments or its excipients or to drugs of similar chemical class
   i. Any other medical condition which, in the investigator's opinion, makes the patient unsuitable for the study
7. Contraception provisions:
   Females: Due to the teratogenic potential of LEN, FCBP must:
   Applicable in all countries except the US:
a. Not be pregnant as confirmed by a negative serum pregnancy test at screening and a medically supervised urine pregnancy test prior to starting study therapy
b. Refrain from breast feeding and donating oocytes during the course of study and for 3 months after the last dose of study drug or according to local guidelines for LEN, whichever is longer
c. Agree to ongoing pregnancy testing during the course of the study, and after study therapy has ended. This applies even if the patient applies complete sexual abstinence
d. Commit to continued abstinence from heterosexual intercourse if it is in accordance with her lifestyle (which must be reviewed on a monthly basis) or agree to use and be able to comply with the use of highly effective contraception without interruption at least 4 weeks prior to start of study drugs, during the study treatment and for 3 months after the last dose of study drug, or, for LEN, according to the local guidelines, whichever is longer
Applicable in the US:
e. Not be pregnant as confirmed by pregnancy tests performed before treatment initiation, within 10-14 days and again within 24 hours of initiating treatment (even if true abstinence is the chosen method of birth control)
f. Refrain from breast feeding and donating oocytes during the course of the study and for 3 months after the last dose of study drug, or according to US guidelines for LEN, whichever is longer
g. Agree to ongoing pregnancy testing during the course of the study (every 3 weeks in women with regular menstrual cycle and every two weeks in women with irregular menstrual cycle), and after study therapy has ended (even if true abstinence is the chosen method of birth control)
h. Not get pregnant while taking the study treatment and for at least 3 months after the last dose of study treatments by using at the same time 2 effective methods of contraception, at least one highly effective method and one additional effective method, each time engaging in sexual activity with a male, starting at least 4 weeks before taking the study treatment, while taking the study treatment, during breaks (dose interruptions) and for at least 3 months after stopping the study treatment, or for LEN, according to the US guidelines, whichever is longer.
True abstinence from heterosexual sexual intercourse is also an acceptable method of contraception.
The use of emergency contraception is also permitted
8. Male participants must:
   Applicable in all countries except the US:
   a. Use an effective barrier method of contraception without interruption if the patient is sexually active with a FCBP. Male patients should refrain from donating sperm during the study participation and for 3 months after the last dose of study treatment, or according to the local guidelines for LEN, whichever is longer
   Applicable in the US:
   b. Use a latex or synthetic condom each time they have sex with a FCBP. True abstinence from heterosexual sexual intercourse is also an acceptable method of contraception. The use of emergency contraception is also permitted. Male patients should refrain from donating sperm during the study participation and for 3 months after the last dose of study drug, or according to the US guidelines for LEN, whichever is longer

### Statistical Analysis

A Primary analysis will be performed when all enrolled patients have either completed C3D28 or discontinued the study prior to C3D28 for any reason. Final analysis will be performed at the end of the study.

Any deviations from the statistical analysis outlined in this protocol will be described, and reasons for the deviations listed, in the clinical study report.

Details of the analyses to be performed on data from this study will be provided in a separate SAP.

### End of Study

The end of the study is defined as the date when the last patient has completed last visit (approximately 3 years after the last patient received the first study treatment).

Upon study closure, MorphoSys will notify the applicable regulatory agencies in accordance with local requirements.

End of study visit for a patient: An end of study visit for a patient is defined as the visit taking place when the patient has completed 90-day safety follow up after the last tafasitamab dose given.

### Appendix 3: Study Specific Definitions

For the purposes of this protocol, primary refractory disease is defined as a disease progressing in the course of the first line treatment as per International Working Group response criteria (Cheson et al., 2007), and/or, showing a response of less than a PR to first-line treatment or disease recurrence/progression within <6 months from the completion of first-line therapy.
Disease refractory to last treatment is defined as having had less than a PR to the most recently administered systemic therapy.
Relapsed/progressive/recurrent disease reflects the appearance of any new lesions or increase by ≥50% of previously involved sites from nadir according to the International Working Group response criteria (Cheson et al., 2007), after the most recent systemic therapy.
End of Treatment: The end of treatment is defined as the date when the patient has received last tafasitamab dose. An end of treatment visit will be performed within 14 days after decision on treatment discontinuation.
End of Study: The end of the study is defined as the date when the last patient has completed last visit (approximately 3 years after the last patient received the first study treatment).
End of study visit for a patient: An end of study visit for a patient is defined as when the patient has completed 90 day safety follow up after the last tafasitamab dose given.
Beginning of study: Start of a clinical study’ means the first act of recruitment of a potential subject for a specific clinical study, unless defined differently in the protocol.

### Appendix 7: Covid-19: Infection Prophylaxis and Vaccines

Live vaccines must not be administered to patients in this study. Killed, inactivated vaccines, such as an injectable annual influenza vaccine, are permitted. Investigators should follow institutional guidelines concerning infection chemoprophylaxis for patients regarded to be at high risk for infection.

Whenever possible, relapsed/refractory patients who will be treated with immunosuppressive therapy including tafasitamab-containing regimens should start vaccination against COVID-19 as soon as possible, at least first dose, ideally approximately 2 weeks prior to study treatment start.

Based on current safety/benefit considerations and in the absence of data or guidance to the contrary, we recommend that all patients with lymphoma should receive a COVID-19 vaccine (unless explicitly contraindicated), accepting that this might not achieve full protection due to the impaired humoral and/or cellular immunity.

For patients who are already on tafasitamab-containing regimens, the advantages and disadvantages of delaying vaccination to allow immune recovery or interrupting therapy requires careful consideration on a case-by-case basis. The recommendation would be to vaccinate these patients despite the fact that may be unable to generate a fully protective immune response to a COVID-19 vaccine.

### Appendix 8: Hepatitis Virus Serology

Patients will be examined according to the SoA for viral hepatitis B and C serology. Hepatitis B biomarkers include HbsAg, total anti-hepatitis B core antibody (anti-HBc) and anti-HBsAb).

Patients with a positive test for anti-HBc can only be included if HBV DNA is not detected. In these patients only, HBV DNA should be assessed at various subsequent visits as outlined in the SoA.

In the context of exclusion criteria, seropositive for or active viral infection with HBV means:
HBV surface antigen positive
HBV surface antigen negative, HBV surface antibody positive and/or HBV core antibody positive and detectable viral DNA. Note: Patients who are HBV surface antigen negative and viral DNA negative are eligible
Patients who exhibit the classical vaccination profile of HBV surface antibody positive, HBV core antibody negative, and HBV surface antigen negative are eligible

If HBV-DNA becomes detectable during treatment, patients should be prophylactically treated and followed-up for potential hepatitis B reactivation as per local medical practice or institutional guidelines for CD20 antibodies such as RTX. If the HBV-DNA assay is positive, then patients can only stay in the study if they are assessed by a physician experienced in the treatment of hepatitis B and pre-emptive treatment is initiated, if deemed appropriate, and/or according to local practice/guidelines.

Hepatitis C serology is to be done at screening only. Hepatitis C biomarkers include anti-HCV antibody. For patients who are positive for anti-HCV antibody, HCV-RNA should be measured.

1. A positive Hepatitis C test is defined as a positive test for HCV antibodies and a positive test for HCV RNA.

1-23. (canceled)

24: A method of treating a cancer in a human subject in need thereof, the method comprising administering to the human subject an anti-CD19 antibody, wherein the anti-CD19 antibody is administered at a dose of at least 24 mg/kg.

25: The method of claim 24, wherein the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

26: The method of claim 24, wherein the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

27: The method of claim 24, wherein the anti-CD19 antibody comprises a heavy chain variable region of
(SEQ ID NO: 7)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSS
and a light chain variable region of
(SEQ ID NO: 8)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIK.

28: The method of claim 24, wherein the anti-CD19 antibody comprises an Fc domain comprising an amino acid substitution at position S239 and/or 1332, wherein the numbering is according to the EU index as in Kabat.

29: The method of claim 24, wherein the anti-CD19 antibody comprises an Fc domain comprising an S239D amino acid substitution and an I332E amino acid substitution, wherein the numbering is according to the EU index as in Kabat.

30: The method of claim 24, wherein the anti-CD19 antibody comprises a heavy chain constant region of
(SEQ ID NO: 9)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPDVFLFPPKPKDTLMISRTPEVTCVVV
DVSHEDPEVQFNWYVDGVEVHNAKTKPREEQFNSTFRVVSVLTVVHQDW
LNGKEYKCKVSNKALPAPEEKTISKTKGQPREPQVYTLPPSREEMTKNQ
VSLTCLVKGFYPSDIAVEWESNGQPENNYKTTPPMLDSDGSFFLYSKLT
VDKSRWQQGNVFSCSVMHEALHNHYTQKSLSLSPGK
and a light chain constant region of
(SEQ ID NO: 10)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

31: The method of claim 24, wherein the anti-CD19 antibody comprises a heavy chain region of
(SEQ ID NO: 11)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPDVF
LFPPKPKDTLMISRTPEVTCVVVDVSHEDPEVQFNWYVDGVEVHNAKTK
PREEQFNSTFRVVSVLTVVHQDWLNGKEYKCKVSNKALPAPEEKTISKT
KGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYT
QKSLSLSPGK
and a light chain region of
(SEQ ID NO: 12)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

32: The method of claim 24, wherein the anti-CD19 antibody is administered intravenously, by intravenous infusion or subcutaneously.

33: The method claim 24, wherein said dose reduces the dosing frequency from once weekly to at least once every two weeks.

34: The method of claim 24, wherein the cancer is a hematological malignancy.

35: The method of claim 24, wherein the cancer is a lymphoma or a leukemia.

36: The method claim 24, wherein the cancer is non-Hodgkin's lymphoma (NHL), chronic lymphocytic leukemia (CLL), small lymphocytic lymphoma (SLL) or acute lymphoblastic leukemia (ALL).

37: The method of claim 24, wherein the cancer is Diffuse large B-cell lymphoma (DLBCL) or relapsed or refractory Diffuse large B-cell lymphoma (r/r DLBCL).

38: The method of claim 24, wherein the anti-CD19 antibody is administered once a week, once every two weeks or once every four weeks.

39: The method of claim 24, wherein the anti-CD19 antibody is administered at a dose in the range of between 24 mg/kg to 30 mg/kg.

40: The method of any claim 24, wherein the anti-CD19 antibody is administered at a dose of 24 mg/kg.

41: The method of claim 24, wherein the anti-CD19 antibody is administered at a dose of 30 mg/kg.

42: The method of claim 24, wherein the anti-CD19 antibody is administered in 28-day cycles, wherein on: a) days 1, 4 and 9 of the first cycle, a dose of 12 mg/kg is administered and on day 15 of the first cycle a dose of at least 24 mg/kg is administered; b) days 1 and 15 of cycles 2-3, a dose of at least 24 mg/kg is administered; and c) day 1 of further subsequent cycles, a dose of at least 24 mg/kg is administered.

43: The method of claim 24, wherein the anti-CD19 antibody is administered in 28-day cycles, wherein on: a) days 1, 4 and 9 of the first cycle, a dose of 12 mg/kg is administered and on day 15 of the first cycle a dose in the range of between 24 mg/kg to 30 mg/kg is administered; b) days 1 and 15 of cycles 2-3, a dose in the range of between 24 mg/kg to 30 mg/kg is administered; and c) day 1 of further subsequent cycles, a dose in the range of between 24 mg/kg to 30 mg/kg is administered.

44: The method of claim 24, wherein the anti-CD19 antibody is administered in 28-day cycles, wherein on: a) days 1, 4 and 9 of the first cycle, a dose of 12 mg/kg is administered and on day 15 of the first cycle a dose of 24 mg/kg is administered; b) days 1 and 15 of cycles 2-3, a dose of 24 mg/kg is administered; and c) day 1 of further subsequent cycles, a dose of 24 mg/kg is administered.

45: The method of claim 24, wherein the anti-CD19 antibody is administered in 28-day cycles, wherein on: a) days 1, 4 and 9 of the first cycle, a dose of 12 mg/kg is administered and on day 15 of the first cycle a dose of 30 mg/kg is administered; b) days 1 and 15 of cycles 2-3, a dose of 30 mg/kg is administered; and c) day 1 of further subsequent cycles, a dose of 30 mg/kg is administered.

46: The method of claim 24, wherein the anti-CD19 antibody is administered in combination with lenalidomide.


SEQUENCE LISTING (13 sequences)

SEQ ID NO: 1 - protein, 5 aa, synthetic construct
  REGION 1..5: note = source = /note="Description of Artificial Sequence: Synthetic peptide"
SYVMH

SEQ ID NO: 2 - protein, 6 aa, synthetic construct
  REGION 1..6: note = source = /note="Description of Artificial Sequence: Synthetic peptide"
NPYNDG

SEQ ID NO: 3 - protein, 12 aa, synthetic construct
  REGION 1..12: note = source = /note="Description of Artificial Sequence: Synthetic peptide"
GTYYYGTRVFDY

SEQ ID NO: 4 - protein, 16 aa, synthetic construct
  REGION 1..16: note = source = /note="Description of Artificial Sequence: Synthetic peptide"
RSSKSLQNVNGNTYLY

SEQ ID NO: 5 - protein, 7 aa, synthetic construct
  REGION 1..7: note = source = /note="Description of Artificial Sequence: Synthetic peptide"
RMSNLNS

SEQ ID NO: 6 - protein, 9 aa, synthetic construct
  REGION 1..9: note = source = /note="Description of Artificial Sequence: Synthetic peptide"
MQHLEYPIT

SEQ ID NO: 7 - protein, 121 aa, synthetic construct
  REGION 1..121: note = source = /note="Description of Artificial Sequence: Synthetic polypeptide"
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, synthetic construct
  REGION 1..112: note = source = /note="Description of Artificial Sequence: Synthetic polypeptide"
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, synthetic construct
  REGION 1..330: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 451 aa, synthetic construct
  REGION 1..451: note = source = /note="Description of Artificial Sequence: Synthetic polypeptide"
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, synthetic construct
  REGION 1..219: note = source = /note="Description of Artificial Sequence: Synthetic polypeptide"
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]